(19) United States
(12) Patent Application Publication
(10) Pub. No.: US 2013/0110238 A1
(43) Pub. Date: May 2, 2013

(54) INTERBODY IMPLANT

(71) Applicants: Gary Lindemann, Collierville, TN (US); Paula A. Nichter, Memphis, TN (US)

(72) Inventors: Gary Lindemann, Collierville, TN (US); Paula A. Nichter, Memphis, TN (US)

(73) Assignee: WARSAW ORTHOPEDIC, INC., Warsaw, IN (US)

(21) Appl. No.: 13/282,183

(22) Filed: Oct. 26, 2011

(51) Int. Cl. A61F 2/44

(52) U.S. Cl. 623/17.12

(57) ABSTRACT

An interbody implant is provided. The interbody implant includes a body having a cavity configured to retain material deposited in the cavity. The cavity has an opening defining a passageway in communication with the cavity. The implant further includes a membrane disposed about the opening of the cavity having a first configuration covering the opening and a second configuration such that the membrane exposes the passageway in communication with the cavity.

### TECHNICAL FIELD

The present disclosure generally relates to medical devices for the treatment of musculoskeletal disorders, and more particularly to an interbody implant having a cavity for insertion of material, such as bone growth promoting material.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility. For example, after a disc collapse, severe pain and discomfort can occur due to the pressure exerted on nerves and the spinal column.

Non-surgical treatments, such as medication, rehabilitation and exercise can be effective, however, may fail to relieve the symptoms associated with these disorders. Surgical treatment of these spinal disorders includes fusion, fixation, discectomy, laminectomy and implantable prosthetics. These treatments may employ interbody implants. This disclosure describes improved interbody implants used in surgical treatment of spinal disorders.

### SUMMARY OF THE INVENTION

Accordingly, an interbody implant having a cavity covered by a membrane is provided. The membrane is configured to cover a passageway in communication with the cavity of the implant in a first position, and exposes the passageway in a second position. It is contemplated that the cavity can have two openings so as to extend from one surface of the implant to an opposite second surface connected by a passageway wherein each opening is covered by a membrane. One or both of the membranes is configured so that the membrane can be moved from a first position covering the opening to a second position wherein the passageway is exposed. It is contemplated that material such as, bone growth material, can be placed in the cavity and the membrane positioned to cover the passageway retains the material in the cavity as the implant is inserted into the spine of a patient. The implant of the present disclosure can be inserted in the spine of a patient using minimally invasive and percutaneous techniques.

In one embodiment in accordance with the principles of the present disclosure, an interbody implant is provided which includes a body having a cavity configured to retain material deposited in the cavity. The cavity having an opening defining a passageway in communication with the cavity. The implant further includes a membrane disposed about the opening of the cavity having a first configuration covering the opening and a second configuration such that the membrane exposes the passageway in communication with the cavity. The membrane can be directly molded into the device or can be a separate member attached after the interbody implant is molded.

In another embodiment in accordance with the principles of the present disclosure, an implant having a body including a cavity configured to retain material deposited in the cavity is provided. The cavity in the implant includes an opening defining a passageway in communication with the cavity, and a membrane attached about the opening of the cavity. The membrane comprising an elongated slit configured to bifurcate the membrane into a first portion and a second portion wherein retracting the first and second portions in an opposite direction to one another defines a second position such that at least a portion of the passageway is exposed. In the alternative the membrane can be constructed of two separate membranes configured to at least partially overlap one another along the elongated slit wherein separating the two overlapping membranes exposes the passageway to the cavity.

In yet another embodiment, an interbody implant is provided which includes a body having a cavity configured to retain material deposited in the cavity. The cavity has an opening defining a passageway in communication with the cavity. The implant further includes a membrane comprising an elongated slot having an edge that in a first position defines a gap in the membrane. Retracting the membrane to a second position widens the gap so that material can be inserted into the cavity. The membrane is configured so that it returns back to the first position when released from the retracted position.

In another embodiment in accordance with the principles of the disclosure, a kit comprising at least one implant of the present invention and a discrete amount of at least one material to be inserted into the cavity of the implants prior to insertion into a spine of a patient is provided.

### BRIEF DESCRIPTION OF THE DRAWINGS

The present disclosure will become more readily apparent from the specific description accompanied by the following drawings, in which:

FIG. 1 is a side, cross sectional view of one embodiment of an implant in accordance with the principles of the present disclosure with the membrane in the closed position;

FIG. 2 is a side, cross sectional view of the implant shown in FIG. 1 with the membrane in an open position;

FIG. 3 is a side, cross sectional view of the implant shown in FIG. 1 with the membrane in a fully open position;

FIG. 4 is a perspective view of one embodiment of an implant in accordance with the principles of the present disclosure;

FIG. 5 is a perspective view of the implant shown in FIG. 1 including a membrane in accordance with the principles of the present disclosure with the membrane in a closed position;

FIG. 6 is a perspective view of the implant shown in FIG. 5 with the membrane in an open position;

FIG. 7 is a perspective view of one embodiment of an implant in accordance with the principles of the present disclosure;

FIG. 8 is a perspective view of the implant shown in FIG. 7 including a membrane in accordance with the principles of the present disclosure with the membrane in a closed position;

FIG. 9 is a perspective view of the implant shown in FIG. 8 with the membrane in an open position;

FIG. 10 is a perspective view of an implant in accordance with the principles of the present disclosure with a membrane in a closed position; and

FIG. 11 is a perspective view of the implant shown in FIG. 10 with the membrane in an open position;

Like reference numerals indicate similar parts throughout the figures.

### DETAILED DESCRIPTION OF THE INVENTION

The exemplary embodiments of the interbody implant disclosed are discussed in terms of medical devices for the treatment of musculoskeletal disorders and more particularly, in terms of an interbody implant that includes a membrane configured to cover at least a portion of an opening of a cavity in the implant in a first position, and provides a passageway in and out of the cavity for material, such as bone growth promoting material, in a second position. It is envisioned that the implant may have at least one additional opening defining a passageway that is in communication with the first opening and passageway. The first and second openings may be covered either by a membrane or a segment and/or strip of specialty tape affixed about the first and/or second opening. The tape can be bioreabsorbable and forms a permeable, semipermeable, or impermeable membrane designed to retain material in the cavity of the implant as the implant is installed. In some embodiments, an implant as described herein may be provided in a kit along with a segment and/or roll of such specialty tape such that a surgeon may use the tape to create a bioreabsorbable barrier to selectively retain material in the cavity of the implant as described further herein. It is envisioned that the interbody implant may be employed to treat a variety of spine injuries and/or disorders, such as fusion and fixation treatments to provide decompression and/or restoration of lordosis. In these procedures it is often the case that the surgeon has to hammer the implant into the spine of a patient, often with an pounding force. The membrane covering the cavity in the implant serves to retain the material, such as bone growth factors, in the cavity during placement. This assures the surgeon that the material necessary for proper bone growth around and in-growth into the implant remains in the cavity as the implant is pounded into the spine of the patient.

It is envisioned that the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is contemplated that the interbody implants of the present disclosure may be employed with other osteal and bone related applications, including those associated with diagnostics and therapeutics. It is further contemplated that the disclosed interbody implant may be alternatively employed in a surgical treatment with a patient in a prone or supine position, and/or employ various surgical approaches to the spine, including anterior, posterior, posterior mid-line, medial, lateral, postero-lateral, and/or antero-lateral approaches, and in other body regions. The present disclosure may also be alternatively employed with procedures for treating the lumbar, cervical, thoracic and pelvic regions of a spinal column. The interbody implant of the present disclosure may also be used on animals, bone models and other non-living substrates, such as, for example, in training, testing and demonstration.

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “superior” and “inferior” are relative and used only in the context to the other, and are not necessarily “upper” and “lower”.

Further, as used in the specification and including the appended claims, “treating” or “treatment” of a disease or condition refers to performing a procedure that may include administering one or more drugs to a patient in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

The components of the interbody implant can be fabricated from biologically acceptable materials suitable for medical applications, including metals, synthetic polymers, ceramics, and bone material and/or their composites, allograph material depending on the particular application and/or preference of a medical practitioner. For example, the components of the interbody implant, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of the interbody implant may have material composites, including the above materials, to achieve various desired characteristics such as strength, rigidity, elasticity, compliance, biomechanical performance, durability and radiolucency or imaging preference. The components of the interbody implant, individually or collectively, may also be fabricated from a heterogeneous material such as a combination of two or more of the above-described materials.

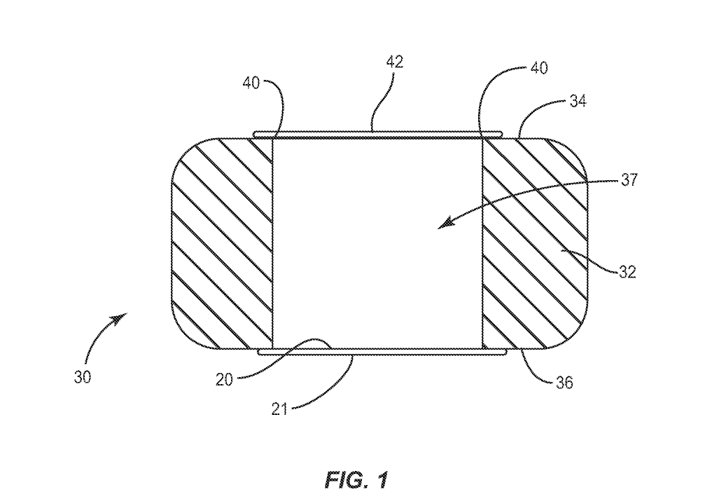

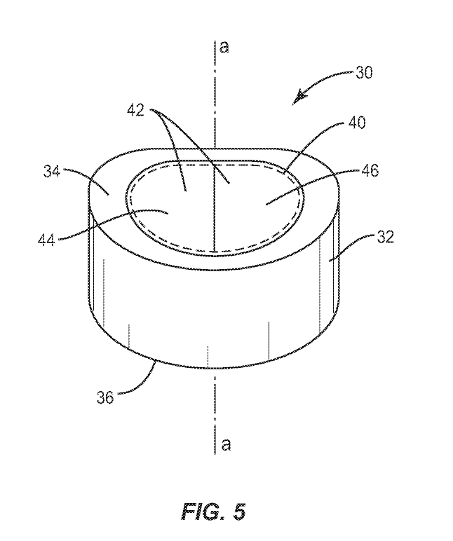

The following discussion includes a description of an interbody implant in accordance with the principles of the present disclosure. Alternate embodiments are also disclosed. Reference will now be made in detail to the exemplary embodiments of the present disclosure, which are illustrated in the accompanying figures. Turning now to FIGS. 1-11, there is illustrated components of the interbody implant in accordance with the principles of the present disclosure.

An interbody implant 30 is employed as a stabilization device in fusion and fixation procedures, for example, for patients suffering from a spinal disorder to provide height restoration between vertebral bodies, decompression and/or restoration of lordosis. Implant 30 may be monolithically formed, integrally connected or include fastening elements and/or instruments, for example, as described herein. Implant 30 is configured such that it can be inserted between adjacent vertebrae and includes a body having a middle portion 32 positioned between upper and lower surfaces 34, 36. Implant 30 has a height defined by the distance between upper and lower surfaces 34, 36 that is approximately the distance between two adjacent vertebral bodies.

Implant 30 includes a cavity 37 configured to retain material, such as bone growth material, for example, deposited therein to promote bonding and/or fusion of implant 30 to adjacent vertebrae. Cavity 37 has a first opening 38 extending through upper surface 34 that defines a passageway in communication with the opening and the cavity. The first opening provides a passageway for placement of the material into cavity 37. It is envisioned that opening 38 may be in the upper or lower surfaces 34, 36. It is envisioned that opening 38 may also be disposed through middle portion 32 and may assume a variety of shapes depending upon, for example, the size and shape of cavity 37, the region of the spine in which implant 30 is inserted between and/or the type of material placed therein. Upper surface 34 has an edge 40 that defines the circumference of opening 38.

Implant 30 includes a membrane 42 covering at least a portion of first opening 38 and is attached around at least part of the opening in one embodiment and substantially around the entire circumference of first opening 38 along edge 40 in another embodiment of the implant of the present disclosure. Attachment of the membrane about the opening 38 prevents leakage or spillage of materials and/or bone growth through opening 38. Membrane 42 forms a seal with edge 40, to retain material, such as bone graft material, autograft material, allograft material, bone growth promoting material, osteoconductive material such as hydroxyapatite, an osteoinductive agent such as a bone morphogenic protein (BMP) and/or other biologic materials and agents within cavity 37 of implant 30 during placement of implant 30. An adhesive material may be used to create the seal between membrane 42 and at least a portion of edge 40 by adhering the perimeter of membrane 42 along edge 40 to cover at least a portion of opening 38.

It is envisioned that the adhesive can be used to attach membrane 42 to the implant about edge 40 after the material has been placed into cavity 37 of the implant 30 and membrane 42 is returned to the closed position. It is also envisioned that the seal between membrane 42 and edge 40 may prevent the leakage of not only the material placed in the cavity but also liquids, such as bodily fluids including blood and/or gases into the cavity. In one embodiment of the present disclosure, an annular ring is positioned about the circumference of opening 38 along edge 40 and provides a surface to which membrane 42 may adhere. That is, once the annular ring is positioned about the circumference of opening 38 along edge 40, membrane 42 may adhere to the annular ring such that membrane 42 forms a seal with the annular ring to cover opening 38.

Membrane 42 may be permeable, porous, or semi-permeable or semi-porous. A semi-permeable membrane retains the material within cavity 37 as the implant is installed and allows some of the materials within cavity 37 of implant 30 to escape through the semi-permeable membrane 42 over time once the implant is installed. Alternatively, membrane 42 may be fabricated from a material that is impermeable to prevent bone growth or leakage therethrough. Membrane 42 may be fabricated from elastomeric materials such as, for example, mylar, rubber, polyurethane, vinyl, latex, polyethylenes, ionomer, and polytetrapthalate (PET), as well as less flexible materials such as stainless steel, titanium, nickel-titanium alloy, and ceramic mesh or weaved materials and combinations thereof. All or a portion of membrane 42 may be made permeable by fabricating a material, including but not limited to, the membrane materials listed above, into a fabric, weave, mesh, composite, bonded fiber assembly, or any other manufacture known to those skilled in the art. In one embodiment, the area of membrane 42 is equal to or greater than the area of opening 38 such that membrane 42 covers the entire area of opening 38. However, it is also envisioned that membrane 42 may have an area that is less than that of opening 38 such that membrane 42 covers only a portion of opening 38. The membrane can be a separate material or molded directly into the implant.

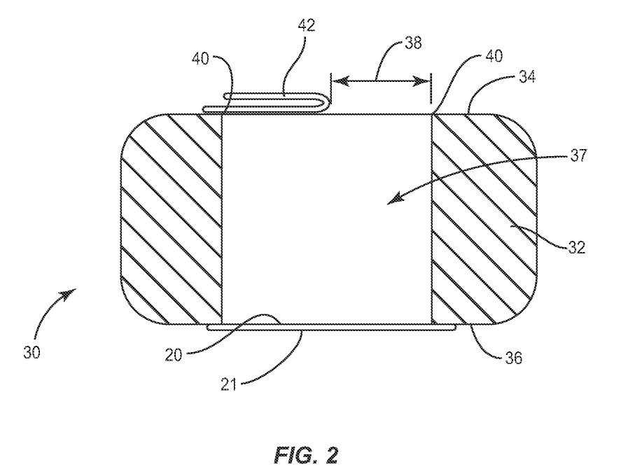

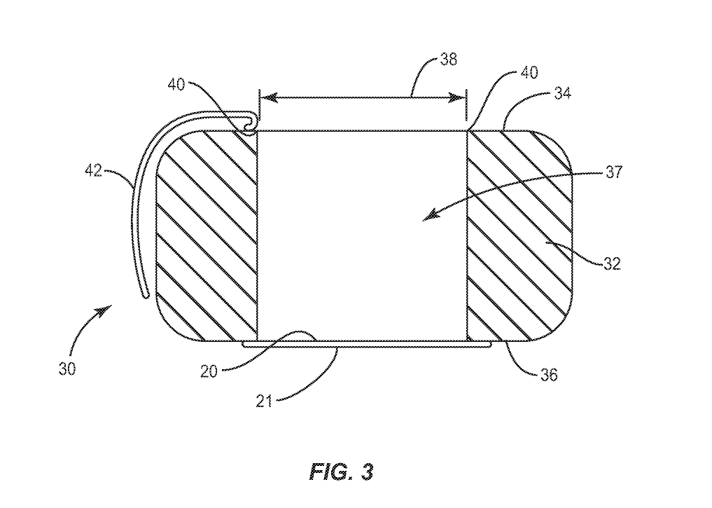

In one embodiment, membrane 42 is movable between a first position, wherein membrane 42 covers at least a portion of opening 38 in cavity 37 as shown in FIG. 1, and a second position, wherein membrane 42 is moved to expose at least a portion of opening 38 in cavity 37 as shown in FIGS. 2 and 3. In the first position, membrane 42 is configured to retain material, such as bone graft material, within cavity 37 of implant 30 by preventing the same from escaping implant 30 through first opening 38 and/or prevents bone growth through opening 38. In the second position, membrane 42 is configured to expose a passageway in communication with cavity 37. Membrane 42 may be moved from the first position to the second position by retracting at least a portion of membrane 42 so as to expose at least a portion of opening 38, as shown in FIGS. 2 and 3, which permits the material to be inserted into the cavity 37 through opening 38. The amount of membrane 42 that is retracted, depends upon, for example, the space necessary for inserting the bone graft material into the cavity and/or the type of material to be inserted into cavity 37 through opening 38. That is, only a small portion of membrane 42 may be refracted, as shown in FIG. 2, if, for example the material to be inserted into cavity 37 is substantially in a powder form. A larger portion of membrane 42 may be refracted, as shown in FIG. 3, if, for example, the material to be inserted into or removed from cavity 37 is not in powder form but instead has a structural form.

It is also envisioned that membrane 42 may be completely removed from implant 30 and placed back onto the implant with adhesive once the material is placed into cavity 37. It is also envisioned that the membrane can be a dispensable tape cut to size prior so as to cover the openings on the implant. The tape can be permeable, semi-permeable or impermeable. The tape can have apertures slots or have a netting mesh configuration. In addition, the tape can be bioabsorbable so as to reabsorb into the body over time, or nonabsorbable so as to be permanent. Material, such as one or more bone grafts, may be inserted into or escape from cavity 37 through opening 38 when membrane 42 is in the second position. Membrane 42 remains refracted to maintain membrane 42 in the second position. At least a portion of membrane 42 may then be repositioned back over opening 38 and adhered to the edge around opening 38 for example, using adhesive, so as to return membrane 42 to the first position. It is envisioned that membrane 42 may fabricated from an elastic or woven material that makes membrane 42 retractable from the first to the second position and back to the second position once material is placed into cavity 37. As stated above. The membrane can be at least partially molded directly into the implant or can be formed separately from the implant and placed on the implant after the implant is formed.

In one embodiment, membrane 42 has at least one aperture extending through membrane 42 that is configured to allow material to pass into and out of cavity 37. The aperture in membrane 42 maybe configured to allow in-growth of bone into and throughout implant 30 when membrane 42 is in the first, closed position. In one embodiment, the aperture in membrane 42 is in communication with cavity 37 of implant 30 and opening 38. That is, the aperture in membrane 42, cavity 37 and opening 38 form a passageway for materials deposited in cavity 37 such that material may enter and/or exit cavity 37 through opening 38 and/or the aperture in membrane 42 allowing material to migrate out of cavity 37 once the implant 30 is set in the spine. The aperture also allows for bone in-growth into the implant 30. In one embodiment, membrane 42 includes a plurality of apertures, such as perforations or mini-slits, extending through the membrane 42.

In another embodiment of the present disclosure, implant 30 includes at least one additional opening 20 defining a passageway that may be in communication with cavity 37 and/or the passageway of the first opening. An additional membrane 21 (or length of tape) can be disposed about the additional opening 20 to cover the additional opening 20. That is, cavity 37 has a first opening 38 in communication with the additional opening 20 such that material deposited in cavity 37 is available from the additional opening 20 through the additional membrane 21. Additional membrane 21 is attached completely about the circumference of the additional opening 20 so as not to open and may be directly molded into the implant. The membrane can serve as a window allowing the surgeon to visualize the material placed in the cavity from a different vantage point than from the first opening 38. This membrane 21 is helpful to the surgeon for making sure that the material placed in cavity 37 prior to placement of the implant into the spine is still in the cavity 37 after it is set in the spine.

In one embodiment, implant 30 includes at least one additional cavity in the body of implant 30 configured to retain material deposited therein. The additional cavity includes at least one additional opening in the body of implant 30 defining at least one additional passageway in communication with the additional cavity. That is, the additional cavity is in communication with the additional opening such that material can be deposited in the additional cavity from the additional opening. At least one additional membrane is disposed about the additional opening. The additional membrane has or may have a first configuration covering the additional opening in the body of implant 30 and a second configuration such that the additional membrane exposes the additional opening or may be sealed closed about the edges. For those cavities in which the membrane is sealed closed about the entire circumference of the opening of the additional cavity, an additional access opening, such as apertures, mini-slits in the membrane is provided. Material can then be added to the additional cavity through these access apertures and during placement of the implant into the spine, the membrane will serve to retain most, if not all of the material in the additional cavity(ies).

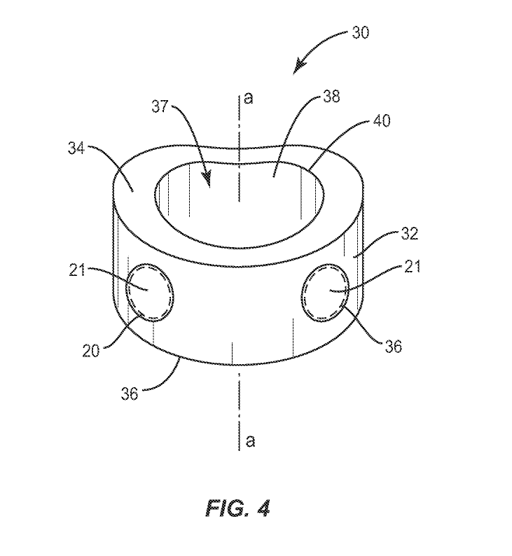

In one embodiment, shown in FIGS. 4-6, membrane 42 is bifurcated into a first portion 44 and a second portion 46 along a line which is parallel to a longitudinal axis a of implant 30. However, it is envisioned that membrane 42 may be bifurcated along a line having angles ranging from 0° to 90° and from 0° to −90° relative to longitudinal axis a of implant 30. First and second portions 44, 46 have ends 45, 47 that are substantially planar, such that ends 45, 47 may converge to form an interface without any gap therebetween. However, it is envisioned that ends 45, 47 may have a shape which is arcuate or irregular, such as saw-toothed, for example, such that the shape of end 45 corresponds to the shape of end 47, and vice versa, such that ends 45, 47 converge to form an interface without any gap therebetween. It is also envisioned in one embodiment, that ends 45, 47 are configured so as to overlap one another and do not form a gap between the edges in a first position. First and second portions 44, 46 have the same size and shape. However, it is envisioned that first portion 44 may have a larger area than second portion 46, or vice versa.

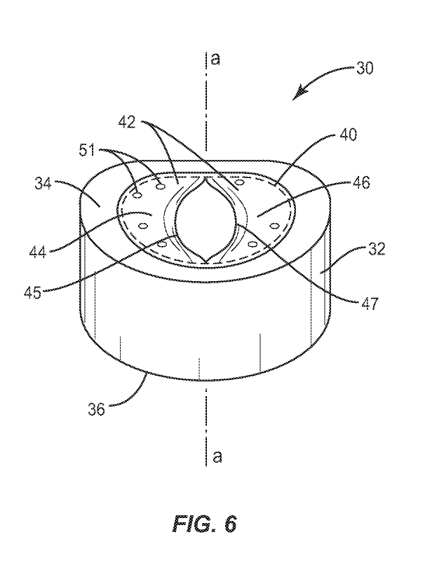

As discussed above, when membrane 42 is in the first position, ends 45, 47 of first and second portions 44, 46 converge or overlap to form an interface such that there is no gap between ends 45, 47. It is envisioned that first and second portions 44, 46 may engage to form a seal that may prevent the passage of materials, such as powdered materials and fluids, for example, therethrough. Membrane 42 may be moved from the first position to the second position by retracting first and second portions 44, 46 of membrane 42 in opposite directions so as to create a passageway 48 between first and second portions 44, 46, that is in communication with the cavity 37 as shown in FIG. 6. That is, passageway 48, cavity 37 in implant 30 and opening 38 are in communication with one another so that material can be placed in or exit cavity 37. Passageway 48 has an area which is less than or equal to the area of opening 38 in cavity 37. Material, such bone grafts, may be inserted into cavity 37 of implant 30 through opening 38 via passageway 48 of membrane 42 when membrane 42 is in the second position. First and second portions 44, 46 can remain retracted to maintain membrane 42 in the second position or can be held to remain in the second position by retracting tools depending on the type of material from which membrane 42 is constructed. That is, membrane 42 may be fabricated from an elastomeric material such that membrane 42 may return to the first position by withdrawing the force used to retract first and/or second portions 44, 46 of membrane 42 in opposite directions so as to cause passageway 48 to close and return to the first, closed position.

In one embodiment, membrane 42 has at least one aperture 51 in first portion 44 and/or second portion 46 that is/are configured to allow small amounts of material to pass into and out of cavity 37 when membrane 42 is in the first, closed position. That is, the aperture(s) 51 in first portion 44 and/or second portion 46 form a mini passageway for materials to enter and/or exit cavity 37. Apertures(s) 51 can be in the form of mini-slits or perforations in the membrane that allow material, such as bone growth material, to escape from cavity 37 to the area surrounding the implant 30. This facilitates bone growth around the implant and also provides for areas of in-growth into the implant 30.

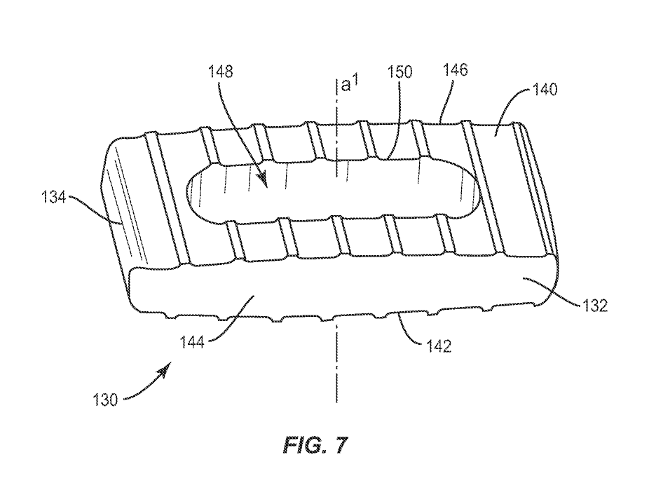

In one embodiment, illustrated in FIGS. 7-9, an implant 130 is provided having a configuration similar to implant 30 which includes a body having a first end, such as proximal end 132, a second end, such as distal end 134 is opposite proximal end 132, upper and lower surfaces 140, 142, and side surfaces 144, 146. Implant 130 includes a cavity 147 (not shown) configured to retain material deposited therein. Cavity 147 has a first opening 148 extending through upper surface 140 that allows the material in cavity 147 to exit via a passageway so as to promote bonding and/or fusion of implant 130 to adjacent vertebrae. It is envisioned that opening 148 may also extend through proximal end 132, distal end 134, side surface 144 or side surface 146 without extending through any other surface of implant 130. Upper surface 140 has an edge 150 about the circumference of opening 148.

Implant 130 includes a membrane 152 covering opening 148 in cavity 147 that is attached about the circumference of opening 148 along edge 150. Membrane 152 is bifurcated into a first portion 154 and a second portion 156 along a line that is perpendicular to a transverse axis a¹ of implant 130. However, it is envisioned that membrane 152 may be bifurcated along a line having angles ranging from 0° to 90° and from 0° to −90° relative to transverse axis a¹ of implant 130. In one embodiment, first and second portions 154, 156 have the same size and shape. However, it is envisioned that first portion 154 may have a larger area than second portion 156, or vice versa. It is also envisioned that first and second portions of the membrane are configured to overlap one another.

Membrane 152 forms a seal with edge 150, to retain material, such as bone graft material, within cavity 147 of implant 130 by preventing the same from escaping from cavity 147 through opening 148. A material, such as an adhesive, for example, may be used to create the seal between membrane 152 and edge 150 by adhering membrane 152 along edge 150 to cover at least a portion of opening 148. It is envisioned that the seal between membrane 152 and edge 150 may prevent the passage of liquids and/or gases from/into cavity 147 of implant 130 through opening 148. The area of membrane 152 is equal to or greater than the area of opening 148 such that membrane 152 covers the entire area of opening 148.

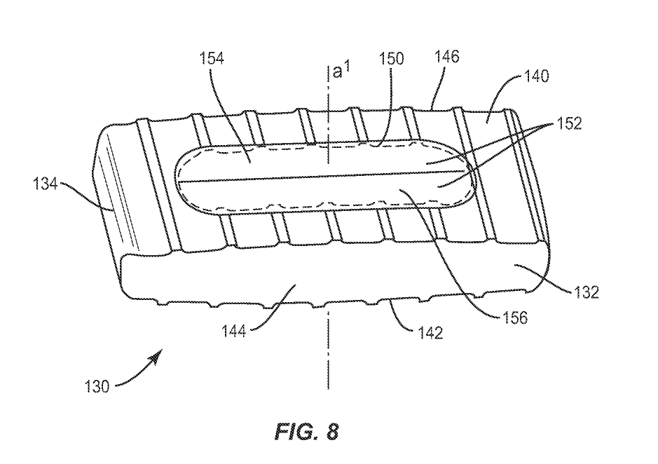

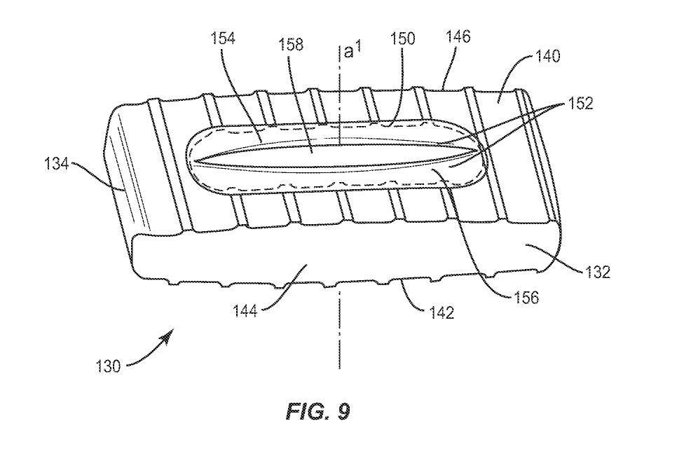

Membrane 152 is movable between a first position, wherein membrane 152 is closed such that membrane 152 covers at least a portion of opening 148 in cavity 147 as shown in FIG. 8, and a second position, wherein membrane 152 is moved to expose or uncover at least a portion of opening 148 in cavity 147 as shown in FIG. 9. In the first position, membrane 152 is configured to retain material within cavity 147 of implant 130 by preventing the same from being displaced from cavity 147 through opening 148 and/or prevent bone growth through implant 130. In the second position, membrane 152 is configured to provide a passageway for material to be inserted into cavity 147 of implant 130 through opening 148. When membrane 152 is in the first position, first and second portions 154, 156 of membrane 152 engage one another such that there is no gap between first and second portions 154, 156. Membrane 152 may be moved from the first position to the second position by retracting first and second portions 154, 156 of membrane 152 in opposite directions so as to create a gap 158, as shown in FIG. 9. Gap 158 has an area which is less than or equal to the area of opening 148 in cavity 147. As with the other embodiments, dispensable tape can be cut to size and used to cover the first opening.

Material, such as bone growth promoting material, may then be inserted into cavity 147 through opening 148 via gap 158 in membrane 152 when in the second, open position. Gap 158, cavity 147 and opening 148 are in communication with one another to form a passageway for material to enter and/or exit cavity 147. Membrane 152 can be fabricated from an elastomeric material such that membrane 152 may return to the first, closed position by withdrawing the force used to retract first and second portions 154, 156 of membrane 152.

In one embodiment, implant 130 includes at least one additional opening in the body of implant 130 defining a passageway that is in communication with cavity 147. The additional opening having a membrane disposed about the entire circumference of the additional opening. The membrane providing a barrier to retain material within the cavity as the implant is installed into the spine. The membrane can also be used as a window to see into cavity 147. This window allows the surgeon to confirm that the material placed within the cavity prior to placing the implant into the spine is still present in the cavity when the implant is partially or fully seated in the spine.

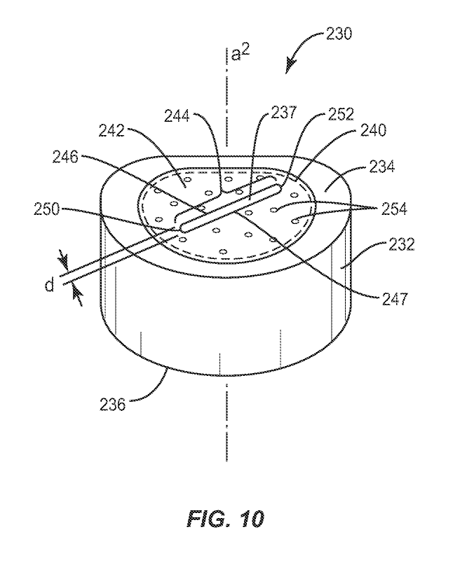

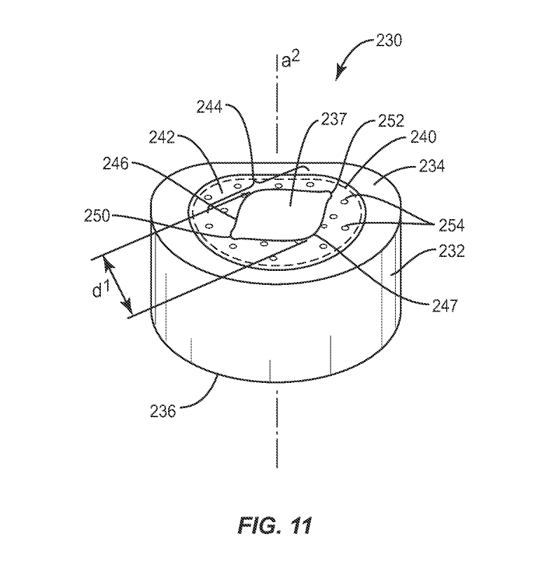

In one embodiment, illustrated in FIGS. 10 and 11, an implant 230 is provided having a configuration similar to implants 30 and 130, and having a body that includes a middle portion 232 positioned between upper and lower surfaces 234, 236. Implant 230 has a height defined by the distance between upper and lower surfaces 234, 236 which is approximately the distance between two adjacent vertebral bodies. Implant 230 includes a cavity 247 (not shown) configured to retain material deposited therein to promote bonding and/or fusion of implant 230 to adjacent vertebrae. Cavity 247 has an opening 237 extending through upper surface 234 and provides a passageway for placement of materials into cavity 247 of implant 230.

Implant 230 includes a membrane 242 configured to cover at least a portion of opening 237 that is adhered to the entire circumference of opening 237 along edge 240. Membrane 242 forms a seal with edge 240, to retain material, such as autograft material, allograft material and/or other biological materials and agents within cavity 247 of implant 230 during placement of implant 230 into the spine of a patient. The membrane 242 prevents the material from becoming dislodged due to the pounding force often associated in setting the implant into the spine of a patient. An adhesive material may be used to create a seal between membrane 242 and edge 240 by adhering membrane 242 along edge 240 to cover opening 237. In one embodiment, an annular ring is positioned throughout the circumference of opening 237 along edge 240 and provides a surface to which membrane 242 may adhere. That is, once the annular ring is positioned about the circumference of opening 237 along edge 240, membrane 242 may adhere to the annular ring such that membrane 242 forms a seal with the annular ring to cover at least a portion of opening 237. The annular ring may be formed directly into the interbody implant or may be attached to the surface of the implant using other methods for attachment known in the art.

Membrane 242 includes an elongate slot 244 extending through membrane 242 along a line that is at an angle between 30° and 60° relative to a longitudinal axis a² of implant 230. However, it is envisioned that elongate slot 244 may extend through membrane 242 along a line having angles ranging from 0° to 90° and from 0° to −90° relative to longitudinal axis a² of implant 230. Elongate slot 244 includes a top portion 246 and a bottom portion 248 opposite top portion 246. In one embodiment, top and bottom portions 246, 248 are planar, parallel to one another and are joined together by convex end portions 250, 252. It is envisioned that elongate slot 244 may assume a variety of shapes depending upon, among other things, the type of material to be placed within implant 230 and/or the configuration of an instrument used to insert the material within implant 230. For example, top portion 246 and/or bottom portion 248 may be convexly or concavely curved.

Membrane 242 is movable between a first position, wherein elongate slot 244 is in its original, non-expanded configuration, as shown in FIG. 10, and a second position, wherein elongate slot 244 is moved to its expanded configuration, enlarging elongate slot 244, as shown in FIG. 11. In the first position, membrane 242 is configured to retain material within cavity 247 of implant 230 by preventing the same from being displaced from cavity 247 through opening 237 during installation. In the second position, membrane 242 is configured to provide a passageway for material to be inserted into cavity 247 through elongate slot 244 and opening 237. When membrane 242 is in the first position, top and bottom portions 246, 248 of elongate slot 244 are separated by a first distance d. When membrane 242 is in the second position, top and bottom portions 246, 248 of elongate slot 244 are separated by a second distance d¹; distance d¹ being greater than distance d. Membrane 242 may be moved from the first position to the second position by retracting top and bottom portions 246, 248 of elongate slot 244 in opposite directions so as to increase the distance between top and bottom portions 246, 248 from first distance d to second distance d¹. Similarly, material may then be inserted into cavity 247 through elongate slot 244 and opening 237. The distance elongate slot 244 should be expanded depends upon, for example, the size and shape of the material to be inserted into cavity 247 of implant 230. Top and bottom portions 246, 248 remain refracted to maintain membrane 242 in the second position. Membrane 242 is fabricated from an elastomeric material such that elongate slot 244 may return to its original, non-expanded configuration by withdrawing the force used to retract top and bottom portions 246, 248 of elongate slot 244 in opposite directions such that top and bottom portions 246, 248 of elongate slot 244 are separated by first distance d.

In one embodiment, membrane 242 includes at least one aperture 254 extending therethrough configured to allow certain material to pass into and out of cavity 247 of implant 230 through opening 237 and allow for bone growth into and through implant 230 when membrane 242 is in the first or second position. Aperture(s) 254 may have a variety of sizes and shapes including, for example, oval, oblong, triangular, rectangular, square, polygonal, irregular, uniform and non-uniform.

In one embodiment, implant 230 includes at least one additional opening in the body of implant 230 defining an additional passageway in communication with cavity 247 and at least one additional membrane that is at least partially translucent and is disposed about the additional opening. The at least partially translucent membrane serving as a window for the surgeon to see into the cavity.

In one embodiment, the interbody implant includes an agent, which includes a bone growth promoting material, which may be disposed, packed or layered within, on or about the components and/or surfaces thereof. The bone growth promoting material, such as, for example, bone graft can be a particulate material, which may include an osteoconductive material such as hydroxyapatite and/or an osteoinductive agent such as a bone morphogenic protein (BMP) to enhance bony fixation of the implants discussed hereinabove with the adjacent vertebrae.

It is contemplated that the bone graft may include therapeutic polynucleotides or polypeptides. It is further contemplated that the bone graft may include biocompatible materials, such as, for example, biocompatible metals and/or rigid polymers, such as, titanium elements, metal powders of titanium or titanium compositions, sterile bone materials, such as allograft or xenograft materials, synthetic bone materials such as coral and calcium compositions, such as hydroxyapatite, calcium phosphate and calcium sulfite, biologically active agents, for example, gradual release compositions such as by blending in a bioresorbable polymer that releases the biologically active agent or agents in an appropriate time dependent fashion as the polymer degrades within the patient. Suitable biologically active agents include, for example, BMP, Growth and Differentiation Factors proteins (GDF) and cytokines Spinal implant 30 can be made of radiolucent materials such as polymers. Radiomarkers may be included for identification under x-ray, fluoroscopy, CT or other imaging techniques.

It is envisioned that the agent may include one or a plurality of therapeutic agents and/or pharmacological agents for release, including sustained release, to treat, for example, pain, inflammation and degeneration. The agents may include pharmacological agents, such as, for example, antibiotics, anti-inflammatory drugs including but not limited to steroids, anti-viral and anti-retroviral compounds, therapeutic proteins or peptides, therapeutic nucleic acids (as naked plasmid or a component of an integrating or non-integrating gene therapy vector system), anticancer agents, stem cells, and combinations thereof. Agents that may also be included in he implant include bone matrix material, composite material, structural scaffold support material, engineered bone material and combinations thereof.

The agent may also include analgesics or anesthetics such as acetic acid derivatives, COX-2 selective inhibitors, COX-2 inhibitors, enolic acid derivatives, propionic acid derivatives, salicylic acid derivatives, opioids, opioid/nonopioid combination products, adjuvant analgesics, and general and regional/local anesthetics.

The agent may also include antibiotics such as, for example, amoxicillin, beta-lactamases, aminoglycosides, beta-lactam (glycopeptide), clindamycin, chloramphenicol, cephalosporins, ciprofloxacin, erythromycin, fluoroquinolones, macrolides, metronidazole, penicillins, quinolones, rapamycin, rifampin, streptomycin, sulfonamide, tetracyclines, trimethoprim, trimethoprim-sulfamthoxazole, and vancomycin.

The agent may also include cancer immunosuppressives agents, such as, for example, steroids, cyclosporine, cyclosporine analogs, cyclophosphamide, methylprednisone, prednisone, azathioprine, FK-506, 15-deoxyspergualin, prednisolone, methotrexate, thalidomide, methoxsalen, rapamycin, leflunomide, mizoribine (bredinin™), brequinar, deoxyspergualin, and azaspirane (SKF 105685), Orthoclone OKT™ 3 (muromonab-CD3). Sandimmune™, Neoral™, Sangdya™ (cyclosporine), Prograf™ (FK506, tacrolimus), Cellcept™ (mycophenolate motefil, of which the active metabolite is mycophenolic acid), Imuran™ (azathioprine), glucocorticosteroids, adrenocortical steroids such as Deltasone™ (prednisone) and Hydeltrasol™ (prednisolone), Folex™ and Mexate™ (methotrxate), Oxsoralen-Ultra™ (methoxsalen) and Rapamuen™ (sirolimus).

In one embodiment in accordance with the principles of the present disclosure, a kit containing at least one intervertebral implant disclosed herein as well as material to be added to the cavity of the implant prior to installation into the spine of a patient. It is also envisioned that the kit may contain additional intervertebral implants of different sizes as well as tools for inserting the material into the cavity of the implants prior to placement. The tools can be disposable or can be sterilized and used again. The kit can be in a sterilized package or can be packaged so that it can be sterilized prior to use at the hospital. The kit may also include dispensable tape that can be used to cover one or more of the openings defined in the implants. The tape can be permeable, semi-permeable or impermeable and the kit can include lengths of each type. It is also envisioned that the tape may be provided with a cross-hatched, screen, mesh and/or porous configuration. A surgeon may thus be provided with a modifiable length of tape configured to be capable of covering the first opening so as to form a membrane disposed about the opening of the cavity. As described herein, the membrane may be configured to prevent the material deposited in the cavity from emerging from the first opening.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. An implant comprising:
a body having a cavity configured to retain material deposited in the cavity, the cavity including a first opening defining a passageway in communication with the cavity; and
a membrane disposed about the opening of the cavity wherein the membrane has a first configuration covering the opening and a second configuration exposing at least a portion of the passageway in communication with the cavity.

2. The implant of claim 1, wherein the membrane is attached about the opening defining the passageway and the membrane comprises an elongated slit configured to bifurcate the membrane into a first portion and a second portion wherein retracting the first and second portions in an opposite direction to one another defines a second position such that at least a portion of the passageway is exposed.

3. The implant of claim 2, wherein the elongated slit is configured such that at least a portion of the first and second portions of the membrane overlap one another in the first position.

4. The implant of claim 1 further comprising:
at least one additional opening in the implant defining a passageway that is in communication with the cavity and the passageway of the first opening; and
a membrane disposed about the at least one additional opening.

5. The implant of claim 1, wherein the material retained in the cavity is selected from the group consisting of autograft material, allograft material, biological agents, analgesic agents, bone growth promoting material, anticancer agents, diagnostic sensors, stem cells, bone matrix, composite materials, structural materials and combinations thereof

6. The implant of claim 5, further comprising at least one aperture in the membrane configured to allow fluid communication into the implant.

7. The implant of claim 6, wherein the at least one aperture is configured as elongated slits, shaped holes, perforations or a combination thereof.

8. The implant of claim 4, wherein the body of the implant comprises a top surface and a bottom surface separated apart from each other by a side wall, wherein the first opening defining the passageway in communication with the cavity is in the top surface and the at least one additional opening defining a passageway in the side wall in communication with the cavity.

9. The implant of claim 4, wherein the body of the implant comprises a top surface and a bottom surface separated apart from each other by a side wall, wherein the first opening defining the passageway in communication with the cavity is in the top surface and the at least one additional opening in the bottom surface defining a passageway in communication with the cavity and the passageway of the first opening.

10. An implant comprising:
a body having a cavity configured to retain material deposited in the cavity, the cavity including a first opening defining a passageway in communication with the cavity; and
a membrane disposed about the opening, the membrane comprising an elongated slot having an outer edge that in a first position defines a gap in the membrane that exposes at least a portion the passageway so that material can be inserted into the cavity through the slot.

11. The implant of claim 10, wherein the membrane is elastomeric and retracting at least a portion of the outer edge away from an opposing portion of the outer edge defines a second position wherein the gap is larger than in the first position and releasing the outer edge of the membrane returns the membrane back to the first position.

12. The implant of claim 11, further comprising:
at least one additional opening in the implant defining a passageway that is in communication with the cavity and the passageway of the first opening.

13. The implant of claim 12 further comprising a second membrane configured to be disposed about the at least one additional opening.

14. The implant of claim 10, wherein the material retained in the cavity is selected from the group consisting of autograft material, allograft material, biological agents, analgesic agents, bone growth promoting material, anticancer agents, diagnostic sensors, stem cells, bone matrix, composite materials, structural materials and combinations thereof

15. The implant of claim 10, further comprising at least one aperture in the membrane so as to provide an area for fluid communication into the implant.

16. The implant of claim 15, wherein the at least one aperture is configured as elongated slits, shaped holes, perforations or a combination thereof.

17. The implant of claim 12, wherein the body of the implant comprises a top surface and a bottom surface separated apart from each other by a side wall, wherein the first opening defining the passageway in communication with the cavity is in the top surface and the at least one additional opening defining a passageway in the side wall in communication with the cavity.

18. The implant of claim 12, wherein the body of the implant comprises a top surface and a bottom surface separated apart from each other by a side wall, wherein the first opening defining the passageway in communication with the cavity is in the top surface and the at least one additional opening in the bottom surface defining a passageway in communication with the cavity and the passageway of the first opening.

19. A surgical kit comprising:
at least one intervertebral implant having a body comprising a cavity configured to retain a graft material deposited in the cavity, the cavity including a first opening defining a passageway in communication with the cavity; and
a length of tape configured to be capable of covering the first opening so as to form a membrane disposed about the opening of the cavity, the membrane being configured to prevent the graft material deposited in the cavity from emerging from the first opening.

20. The surgical kit according to claim 19 further comprising a discrete amount of the graft material selected from the group consisting of autograft material, allograft material, biological agents, analgesic agents, bone growth promoting material, anticancer agents, diagnostic sensors, stem cells, bone matrix, composite materials, structural materials and combinations thereof.

21. The kit according to claim 19 wherein each of the at least one intervertebral implant further comprises at least one additional opening defining a passageway that is in communication with the cavity and the passageway of the first opening and wherein the length of tape is configured to be capable of covering the first and second openings in the at least one intervertebral implant.
